(19) United States
(12) Patent Application Publication
(10) Pub. No.: US 2015/0065704 A1
(43) Pub. Date: Mar. 5, 2015

(54) PROCESS FOR THE PREPARATION AND PURIFICATION OF ESLICARBAZEPINE ACETATE AND INTERMEDIATES THEREOF

(71) Applicants: Ketan Hirpara, Gurgaon (IN); Chandra Has Khanduri, Gurgaon (IN); Mukesh Kumar Sharma, Gurgaon (IN)

(72) Inventors: Ketan Hirpara, Gurgaon (IN); Chandra Has Khanduri, Gurgaon (IN); Mukesh Kumar Sharma, Gurgaon (IN)

(21) Appl. No.: 14/131,823

(22) PCT Filed: Jul. 11, 2012
(86) PCT No.: PCT/IB2012/053550

§ 371 Date: Jul. 24, 2014

(30) Foreign Application Priority Data
Jul. 13, 2011 (IN) 1959/DEL/2011

(51) Int. Cl. C07D 223/28

(52) U.S. Cl. CPC C07D 223/28
USPC 540/589

(57) ABSTRACT

The present invention provides a novel process for the preparation of 10-oxo-10,11-dihydro-5H-dibenzo[b,f]azepine-5-carboxamide, commonly known as oxcarbazepine, which is a medicament and a useful intermediate in the preparation of eslicarbazepine acetate. The present invention further provides a process for the preparation and purification of eslicarbazepine acetate.

### FIELD OF THE INVENTION

The present invention provides a novel process for the preparation of 10-oxo-10,11-dihydro-5H-dibenzo[b,f]azepine-5-carboxamide, commonly known as oxcarbazepine, which is a medicament and a useful intermediate in the preparation of eslicarbazepine acetate. The present invention further provides a process for a preparation and purification of eslicarbazepine acetate.

### BACKGROUND OF THE INVENTION

Eslicarbazepine acetate of Formula A, chemically known as (10S)-5-carbamoyl-10,11-dihydro-5H-dibenzo[b,f]azepin-10-yl acetate is indicated as adjunctive therapy in adults with partial-onset seizures with or without secondary generalisation.

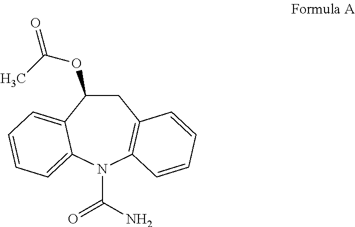 [SMILES: CC(=O)O[C@H]1Cc2ccccc2N(C(N)=O)c2ccccc21]

10-oxo-10,11-dihydro-5H-dibenzo[b,f]azepine-5-carboxamide of Formula 1, commonly known as oxcarbazepine, is an antiepileptic drug marketed under the trade name Trileptal® and is indicated for use as monotherapy or adjunctive therapy in the treatment of partial seizures in adults and as monotherapy in the treatment of partial seizures in children aged 4 years and above with epilepsy, and as adjunctive therapy in children aged 2 years and above with epilepsy. Oxcarbazepine is an intermediate for the preparation of eslicarbazepine.

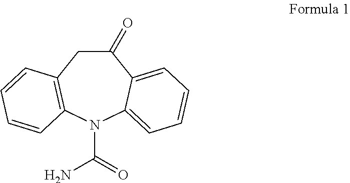 [SMILES: NC(=O)N1c2ccccc2CC(=O)c2ccccc21]

Several processes are known in the literature for making and purifying eslicarbazepine acetate, for example, U.S. Pat. No. 5,753,646; and PCT Publications WO 2006/005951; WO 2007/117166; and WO 2010/113179.

U.S. Pat. No. 5,753,646 provides a process for the preparation of eslicarbazepine acetate which involves adding dropwise a solution of acetyl chloride in dichloromethane to a suspension of (−)-10-hydroxy-10,11-dihydro-5H-dibenz/b,f/azepine-5-carboxamide in dichloromethane and pyridine at a temperature of less than 10° C. under stirring. The residue obtained after workup was crystallized from a mixture of dichloromethane and ethyl acetate to give the eslicarbazepine acetate as white crystals.

U.S Publication No. 2009/0203902 provides preparation of eslicarbazepine acetate which involves the acylation of (S)-(+)-10,11-dihydro-10-hydroxy-5H-dibenz/b,f/azepine-5-carboxamide with acetic anhydride in presence of 4-(N,N-dimethylamino)pyridine and pyridine in dichloromethane at reflux temperature. The resulting solid obtained after work-up was slurried with isopropanol at reflux to obtain a solution. The solution was cooled to 1° C. to 5° C. and eslicarbazepine acetate was isolated from the reaction mass by filtration followed by washing with isopropanol.

PCT Publication No. WO 2010/113179 provides various purification methods of eslicarbazepine acetate which involve the use of acetonitrile/methyl tertiary butyl ether, tetrahydrofuran/n-hexane, tetrahydrofuran/methyl tertiary butyl methyl ether; tetrahydrofuran, methyl ethyl ketone/n-hexane.

Several processes are known in the literature for making oxcarbazepine, for example, U.S. Pat. Nos. 4,452,738 and 7,459,553; PCT Publication Nos. WO 2010/000196; WO 2008/012837; WO 2007/141798; WO 2006/075925; WO 2005/122671; WO 2005/118550; WO 2005/096709; WO 2005/092862; WO 2005/066133; WO 02/096881; WO 00/55138; and WO 96/21649.

PCT Publication No. WO 02/096881 provides a process for the preparation of oxcarbazepine which involves oxidation of 10,11-dihydro-10-hydroxy-5H-dibenz/b,f/azepine-5-carboxamide with peroxyacetic acid in presence of potassium dichromate adsorbed on silica gel at room temperature.

Japanese Patent Publication No. JP 2004-175761 provides a process for the preparation of oxcarbazepine which involves oxidation of 10,11-dihydro-10-hydroxy-5H-dibenzo[b,f]azepine-5-carboxamide with dimethyl sulfoxide and an activator such as sulfur trioxide-pyridine complex.

Chinese Publication No. CN 101302198 provides a process for the preparation of oxcarbazepine which involves oxidation of 10-hydroxy-10,11-dihydro-5H-dibenzo[b,f]azepine-5-carbonitrile with TEMPO and sodium hypochlorite to provide 10-oxo-10,11-dihydro-5H-dibenzo[b,f]azepine-5-carbonitrile which was further hydrolysed with sulfuric acid to obtain oxcarbazepine.

The present inventors have developed an improved process for the preparation of eslicarbazepine acetate and oxcarbazepine which avoids the use of environmentally hazardous reagents and solvents. The present invention further provides eslicarbazepine acetate in good yield, excellent chemical and enantiomeric purity. The process of the present invention avoids excess usage of reagent(s) and organic solvent(s), thereby promoting green chemistry and ensuring a cleaner surrounding by putting lesser load on environment.

### SUMMARY OF THE INVENTION

One aspect of the present invention provides a process for the preparation of oxcarbazepine of Formula 1,

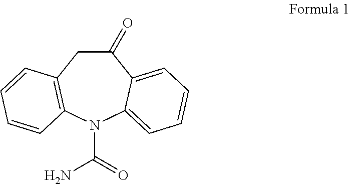 [SMILES: NC(=O)N1c2ccccc2CC(=O)c2ccccc21]

which comprises oxidation of 10-hydroxy-10,11-dihydro-5H-dibenzo[b,f]azepine-5-carboxamide of Formula 2;

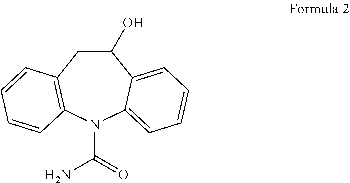 [SMILES: NC(=O)N1c2ccccc2CC(O)c2ccccc21]

with an oxidizing agent, wherein the oxidizing agent is a mixture of (2,2,6,6-Tetramethyl-piperidin-1-yl)oxyl (herein after “TEMPO”) and sodium hypochlorite.

Another aspect of the present invention provides a process for the preparation of eslicarbazepine acetate of Formula A;

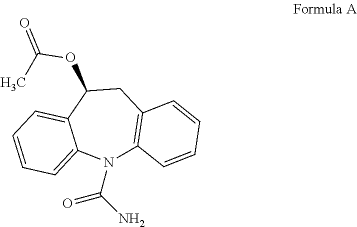 [SMILES: CC(=O)O[C@H]1Cc2ccccc2N(C(N)=O)c2ccccc21]

which comprises:
a) providing a solution of eslicarbazepine acetate in dichloromethane;
b) combining the solution obtained in step a) with a solvent selected from the group consisting of cyclohexane, hexane, toluene, ethyl acetate or a mixture thereof; and
c) isolating eslicarbazepine acetate.

Still another aspect of the present invention provides a process for the preparation of eslicarbazepine acetate of Formula A;

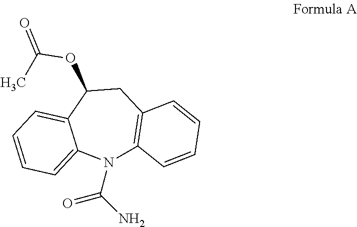 [SMILES: CC(=O)O[C@H]1Cc2ccccc2N(C(N)=O)c2ccccc21]

which comprises:
a) providing a mixture of eslicarbazepine acetate in acetone;
b) combining the mixture obtained in step a) with water; and
c) isolating eslicarbazepine acetate.

Yet another aspect of the present invention provides a process for the preparation of eslicarbazepine acetate of Formula A;

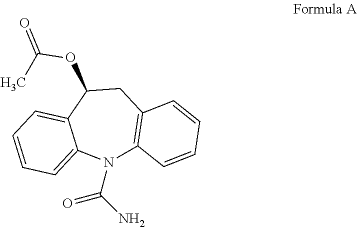 [SMILES: CC(=O)O[C@H]1Cc2ccccc2N(C(N)=O)c2ccccc21]

which comprises:
a) treating (10S)-10-hydroxy-10,11-dihydro-5H-dibenzo[b,f]azepine-5-carboxamide with an acylating agent in acetone;
b) combining the mixture obtained in step a) with water; and
c) isolating eslicarbazepine acetate.

### DETAILED DESCRIPTION OF THE INVENTION

One aspect of the present invention provides a process for the preparation of oxcarbazepine of Formula 1;

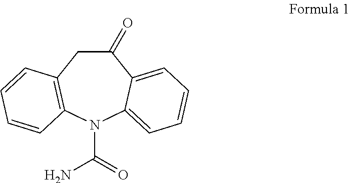 [SMILES: NC(=O)N1c2ccccc2CC(=O)c2ccccc21]

which comprises oxidation of 10-hydroxy-10,11-dihydro-5H-dibenzo[b,f]azepine-5-carboxamide of Formula 2;

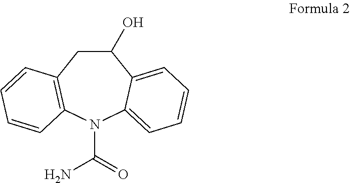 [SMILES: NC(=O)N1c2ccccc2CC(O)c2ccccc21]

with an oxidizing agent, wherein the oxidizing agent is a mixture of TEMPO and sodium hypochlorite.

Oxidation of 10-hydroxy-10,11-dihydro-5H-dibenzo[b,f]azepine-5-carboxamide of Formula 2 with a mixture of TEMPO and sodium hypochlorite may be performed preferably in one or more of solvents.

The term “solvents” includes any solvent or solvent mixture, including for example, water, esters, halogenated hydrocarbons, ketones, ethers, polar aprotic solvents, or mixtures thereof.

The esters may include one or more of ethyl acetate, n-propyl acetate, isopropyl acetate, and n-butyl acetate. Examples of halogenated hydrocarbons include dichloromethane, chloroform, and 1,2-dichloroethane. Examples of ketones include acetone, methyl ethyl ketone and the like. Examples of ethers include diethyl ether, tetrahydrofuran, and the like. A suitable polar aprotic solvent includes one or more of N,N-dimethylformamide, N,N-dimethylacetamide, dimethylsulphoxide, acetonitrile and N-methylpyrrolidone.

Oxidation of 10-hydroxy-10,11-dihydro-5H-dibenzo[b,f]azepine-5-carboxamide of Formula 2 with a mixture of TEMPO and sodium hypochlorite can be performed at a temperature of 0° C. to 50° C. for a time period sufficient to complete the reaction.

After the completion of the reaction, oxcarbazepine can be isolated by common isolation techniques such as extraction, crystallization, precipitation, filtration, decantation, centrifugation, or a combination thereof.

The isolated oxcarbazepine may be further purified by salt formation, crystallization, chromatographic methods, or a combination thereof.

The oxcarbazepine thus obtained according to the present invention may be used as an Active Pharmaceutical Ingredient (API) and may be formulated into finished pharmaceutical products or can be converted to eslicarbazepine or eslicarbazepine acetate by methods exemplified herein or methods known in the art.

Another aspect of the present invention provides a process for the preparation of eslicarbazepine acetate of Formula A;

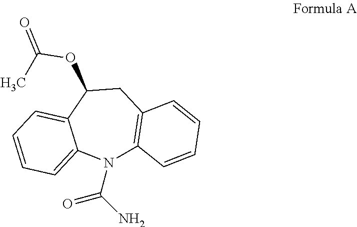 [SMILES: CC(=O)O[C@H]1Cc2ccccc2N(C(N)=O)c2ccccc21]

which comprises:
a) providing a solution of eslicarbazepine acetate in dichloromethane;
b) combining the solution obtained in step a) with a solvent selected from the group consisting of cyclohexane, hexane, toluene, ethyl acetate or a mixture thereof; and
c) isolating eslicarbazepine acetate.

Eslicarbazepine acetate obtained by methods exemplified herein or methods known in the art can be used as the starting material. Methods for preparing eslicarbazepine acetate are well known, e.g., U.S. Pat. No. 5,753,646; and PCT Publication Nos. WO 2007/117166 and WO 2006/005951.

Step a) of providing a solution of eslicarbazepine acetate in dichloromethane includes dissolving eslicarbazepine acetate in dichloromethane, or obtaining an existing solution from a previous processing step of eslicarbazepine acetate in dichloromethane.

The volume of dichloromethane may be about 2 times to about 15 times, preferably 3 times to 10 times, more preferably 3 times to 6 times more than the weight of eslicarbazepine.

The solution obtained in step a) may optionally be filtered in order to remove any extraneous matter present in the solution using any standard filtration techniques known in the art. If desired, a filtering aid such as celite can be added to the solution.

Step b) involves combining the solution obtained in step a) with a solvent selected from the group consisting of cyclohexane, hexane, toluene or a mixture thereof with a mixture of ethyl acetate and hexane. Ethyl acetate and hexane may also be added to the solution obtained in step a) in optional order of succession.

The term “combining” includes dissolving, slurrying, stirring, or a combination thereof.

The solvent can be added at about 10° C. to 20° C., preferably at about 15° C. to 20° C. The solvent can be added for a time period of about 10 minutes to about 90 minutes preferably under stirring.

The volume of solvent may be about 2 times to about 15 times, preferably 3 times to 10 times, more preferably 4 times to 8 times more than the weight of eslicarbazepine and may vary depending on the solvent and the dissolution temperature adopted.

In step c) eslicarbazepine acetate can be isolated by common isolation techniques such as extraction, crystallization, precipitation, filtration, decantation, centrifugation, or a combination thereof.

Still another aspect of the present invention provides a process for the preparation of eslicarbazepine acetate of Formula A;

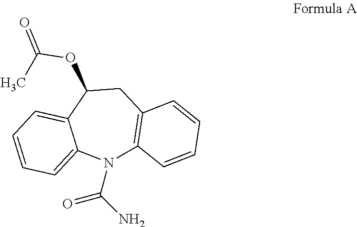 [SMILES: CC(=O)O[C@H]1Cc2ccccc2N(C(N)=O)c2ccccc21]

which comprises:
a) providing a mixture of eslicarbazepine acetate in acetone;
b) combining the mixture obtained in step a) with water; and
c) isolating eslicarbazepine acetate.

Eslicarbazepine acetate obtained by methods exemplified herein or methods known in the art can be used as the starting material. Methods for preparing eslicarbazepine acetate are well known, e.g., U.S. Pat. No. 5,753,646; and PCT Publication Nos. WO 2007/117166 and WO 2006/005951.

Step a) of providing a mixture of eslicarbazepine acetate in acetone includes dissolving or suspending eslicarbazepine acetate in acetone at a temperature of about 25° C. to 40° C. optionally under stirring, or obtaining an existing solution from a previous processing step of eslicarbazepine acetate in acetone.

The mixture obtained in step a) may optionally be filtered in order to remove any extraneous matter present in the solution using any standard filtration techniques known in the art. If desired, a filtering aid such as celite can be added to the solution.

Step b) involves combining the mixture obtained in step a) with water.

The term “combining” includes adding, dissolving, slurrying, stirring, or a combination thereof.

The water can be added at about 10° C. to 40° C., preferably at about 20° C. to 30° C. The water can be added for a time period of about 10 minutes to about 90 minutes, preferably under stirring.

The volume of water may be about 5 times to about 20 times, preferably 10 times to 18 times more than the weight of eslicarbazepine and may vary depending on the dissolution temperature adopted.

In step c) eslicarbazepine acetate can be isolated by any common isolation technique such as cooling, extraction, one or more of washing, crystallization, precipitation, filtration, filtration under vacuum, decantation and centrifugation, or a combination thereof.

Yet another aspect of the present invention provides a process for the preparation of eslicarbazepine acetate of Formula A;

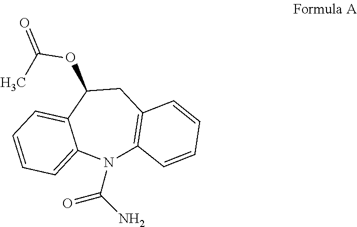 [SMILES: CC(=O)O[C@H]1Cc2ccccc2N(C(N)=O)c2ccccc21]

which comprises:
a) treating (10S)-10-hydroxy-10,11-dihydro-5H-dibenzo[b,f]azepine-5-carboxamide with an acylating agent in acetone;
b) combining the mixture obtained in step a) with water; and
c) isolating eslicarbazepine acetate.

Step a) of treating (10S)-10-hydroxy-10,11-dihydro-5H-dibenzo[b,f]azepine-5-carboxamide with an acylating agent in acetone can be preferably performed in the presence of a catalyst at a temperature of about 20° C. to 40° C. under stirring for a time period sufficient to complete the reaction.

A suitable acylating agent can be selected from the group consisting of acetyl chloride or acetic anhydride. The molar ratio of the acylating agent can be preferably in the range of 0.5 to 1.5.

A suitable catalyst can be selected from the group consisting of organic bases such as 4-dimethylaminopyridine. The molar ratio of the catalyst can be preferably in the range of 0.05 to 0.2.

Step b) involves combining the solution obtained in step a) with water.

The term “combining” includes adding, dissolving, slurrying, stirring, or a combination thereof.

The water can be added at about 10° C. to 40° C., preferably at about 20° C. to 30° C. The water can be added for a time period of about 10 minutes to 90 minutes, preferably under stirring.

The volume of water may be about 5 times to about 20 times, preferably 10 times to 18 times more than the weight of eslicarbazepine and may vary depending on the dissolution temperature adopted.

In step c) eslicarbazepine acetate can be isolated by any common isolation technique such as cooling, extraction, one or more of washing, crystallization, precipitation, filtration, filtration under vacuum, decantation and centrifugation, or a combination thereof.

While the present invention has been described in terms of its specific embodiments, certain modifications and equivalents will be apparent to those skilled in the art and are intended to be included within the scope of the present invention.

### EXAMPLES

### Example 1

### Preparation of 10-Hydroxy-10,11-dihydro-5H-dibenzo[b,f]azepine-5 carboxamide

Sodium borohydride (15.6 g, 0.412 mol) was added to a stirred suspension of oxcarbazepine (200 g, 0.793 mol) in a mixture of methanol (400 mL) and water (220 mL) at 25° C. to 30° C. in three portions at 15 minutes intervals. The reaction mixture was stirred at 60° C. to 65° C. for 2 hours and cooled to 25° C. to 30° C. and pH was adjusted to 6.8 to 7.2 using diluted hydrochloric acid (31 mL concentrated hydrochloric acid in 100 mL deionised water). Deionised water (700 mL) was added to the reaction mixture with stirring and was cooled to 0° C. to 5° C. for 3 hours. The crystalline product was filtered, washed with deionised water (100 mL) and dried at 40° C. to 45° C. to constant weight to give the titled compound.

### Yield: 189.1 g (93.8%)

### Purity: 99.93%

### Example 2

### Preparation of (+) Diacetyl-L-tartaric acid anhydride

L(+) tartaric acid (200 g, 1.33 mol) was stirred with acetic anhydride (530 g, 5.2 mol) at 25° C. to 30° C. and concentrated sulfuric acid (0.9 mL) was added to the mixture. After two minutes, an exothermic reaction started and temperature rose to 80° C. to 85° C. The reaction mixture was then heated at reflux for fifteen minutes, whereupon the volatile components were removed by evaporation at 70° C. to 75° C. under vacuum. Toluene (400 mL) was added to the remaining semisolid mass at 25° C. to 30° C. The reaction mixture was cooled to 10° C. to 15° C. and stirred for 1 hour. The crystalline products were filtered, washed with toluene (70 mL) and dried at 50° C. to 55° C. under vacuum to constant weight to give the titled compound.

### Yield: 246 g (85.4%)

### Example 3

### Preparation of (10S)-10-Hydroxy-10,11-dihydro-5H-dibenzo[b,f]azepine-5-carboxamide tartarate

Pyridine (21 g, 0.265 mol) and 4-dimethylaminopyridine (0.86 g, 0.007 mol) were added to a stirred suspension of racemic 10-hydroxy-10,11-dihydro-5H-dibenzo[b,f]azepine-5-carboxamide (45 g, 0.177 mol) in dichloromethane (450 mL) at 25° C. to 30° C. After stirring at 25° C. to 30° C. for 15 minutes, diacetyl-L-tartaric acid anhydride (47.8 g, 0.221 mol) was added to the mixture. The reaction mixture was stirred at 25° C. to 30° C. for 2 hours whereupon deionised water (315.0 mL) was added dropwise and the resulting reaction mixture was stirred for 4 hours at 15° C. to 20° C. The precipitated solid was filtered off, washed with deionised water (45 mL) and dried at 50° C. to 55° C. to constant weight to obtain the titled compound.

Yield: 37 g (88.9%, based on single diastereisomer)

### Chiral Purity: 90.7%.

### Example 4

### Preparation of (10S)-10-Hydroxy-10,11-dihydro-5H-dibenzo[b,f]azepine-5-carboxamide

3N aqueous sodium hydroxide (22.7 g, 0.568 mol in 190 mL deionised water) was added dropwise to a stirred suspension of (S)-10,11-dihydro-10-hydroxy-5H-dibenzo[b,f]azepine-5-carboxamide tartarate (65 g, 0.138 mol) and methanol (420.0 mL) at 25° C. to 30° C. The reaction mixture was stirred at 25° C. to 30° C. for 45 minutes. The precipitated sodium bitartarate was filtered and washed with methanol (65 mL). The filtrate was concentrated at about 40° C. to 45° C. under reducing pressure. The reaction mixture was cooled to 25° C. to 30° C. and deionised water (325 mL) was added to it. The resulting solution was stirred at 5° C. to 10° C. for 3 hours. The precipitated solid was filtered off, washed with deionised water (65 mL) and dried at 50° C. to 55° C. to constant weight to obtain the titled compound.

### Yield: 30 g (85.4%)

### Chemical Purity: 99.56%

### Chiral Purity: 92.43%

### Example 5

### Preparation of (10S)-10-Hydroxy-10,11-dihydro-5H-dibenzo[b,f]azepine-5 carboxamide

Pyridine (1.1 mL, 0.0126 mol) was added to a stirred suspension of racemic 10-hydroxy-10,11-dihydro-5H-dibenzo[b,f]azepine-5-carboxamide (2.0 g, 0.0079 mol) and ethyl acetate (20 mL) at 30° C. to 35° C. After stirring at 25° C. to 30° C. for 15 minutes, diacetyl-L-tartaric acid anhydride (2.72 g, 0.0126 mol) was added to the mixture. The reaction mixture was stirred at 30° C. to 35° C. for 1 hour whereupon deionised water (20 mL) was added dropwise and the resulting reaction mixture was stirred for 30 minutes at 0° C. to 5° C. The precipitated solid was filtered off, washed with deionised water (10 mL) and dried at 60° C. for 4 hours to obtain (10S)-10-hydroxy-10,11-dihydro-5H-dibenzo[b,f]azepine-5-carboxamide tartarate (Yield: 1.6 g, 86.48%, based on single diastereisomer). 3N aqueous sodium hydroxide (4.8 mL) was added dropwise to a stirred suspension of (10S)-10-hydroxy-10,11-dihydro-5H-dibenzo[b,f]azepine-5-carboxamide tartarate (1.2 g, 0.00255 mol) and methanol (6.0 mL) at 25° C. to 30° C. for 15 minutes. The reaction mixture was stirred at 25° C. to 30° C. for 1 hour. The precipitated sodium bitartarate was filtered and washed with methanol (10 mL). The filtrate was concentrated at about 40° C. to 45° C. under reducing pressure. The reaction mixture was cooled to 25° C. to 30° C. and deionised water (10 mL) was added to it. The resulting solution was stirred at 0° C. to 5° C. for 30 minutes. The precipitated solid was filtered off, washed with deionised water (10 mL) and dried at 60° C. for 4 hours to obtain the titled compound.

### Yield: 0.7 g (77.08%)

### Chiral Purity: 89.17%

### Example 6

### Preparation of Eslicarbazepine Acetate

Acetyl chloride (6.5 g, 0.082 mol) in dichloromethane (50 ml) was added dropwise to a stirred suspension of (10S)-10-hydroxy-10,11-dihydro-5H-dibenzo[b,f]azepine-5-carboxamide (15.0 g, 0.0589 mol) in dichloromethane (250 mL) and pyridine (7.0 g, 0.088 mol) at 15° C. to 20° C. After stirring at 15° C. to 20° C. for 30 minutes, the reaction mixture was further stirred for 2 hours at 25° C. to 30° C. After completion of reaction, the reaction mixture was washed with aqueous sulfuric acid (150 mL), followed by washing with saturated aqueous sodium chloride solution (150 mL) and deionised water (150 mL). The organic layer was divided into three parts by Volume/Volume.

### Part A:

The organic layer obtained above was concentrated to 20 mL at about 35° C. to 40° C. under reduced pressure and was cooled to 15° C. to 20° C. Cyclohexane (15.0 mL) was charged dropwise under stirring. The reaction mixture was stirred for 1 hour at 15° C. to 20° C. The obtained crystalline product was filtered and washed with cyclohexane (5.0 mL). The product was suction dried and then dried under vacuum at 50° C. to 55° C. for 12 hours to obtain the titled compound.

### Yield: 3.0 g (51.5%)

Chemical Purity: 99.86% (Highest individual impurity: 0.03%)

### Chiral Purity: 98.48%

### Part B:

The organic layer obtained above was concentrated to 20 mL at about 35° C. to 40° C. under reduced pressure and was cooled to 15° C. to 20° C. Cyclohexane (25.0 mL) was charged dropwise under stirring. The reaction mixture was stirred for 1 hour at 15° C. to 20° C. The obtained crystalline product was filtered and washed with cyclohexane (5.0 mL). The product was suction dried and then dried under vacuum at 50° C. to 55° C. for 12 hours to obtained titled compound.

### Yield: 4.0 g (68.64%)

Chemical Purity: 99.85% (Highest individual impurity: 0.03%)

### Chiral Purity: 98.64%

### Part C:

The organic layer obtained above was concentrated to 20 mL at about 35° C. to 40° C. under reduced pressure and was cooled to 15° C. to 20° C. Cyclohexane (35.0 mL) was charged dropwise under stirring. The reaction mixture was stirred for 1 hour at 15° C. to 20° C. The obtained crystalline product was filtered and washed with cyclohexane (5.0 mL). The product was suction dried and then dried under vacuum at 50° C. to 55° C. for 12 hours to obtained titled compound.

### Yield: 4.4 g (75.5%)

Chemical Purity: 99.83% (Highest individual impurity: 0.04%)

### Chiral Purity: 98.22%

The results of experiments performed by varying the reaction conditions such as the quantity of solvent (dichloromethane and cyclohexane) for the preparation of eslicarbazepine acetate following the procedure of the above example is summarized in the table below:

| Volume of | Volume of | Purity/ | | |
|---|---|---|---|---|
| dichloromethane | cyclohexane (times | Highest | Chiral | |
| (times with respect | with respect to | individual | Purity | Yield |
| to eslicarbazepine) | eslicarbazepine) | impurity (%) | (%) | (%) |
| 4 | 3 | 99.86 | 98.48 | 51.5 |
| | | 0.03 | | |
| 4 | 5 | 99.85 | 98.64 | 68.64 |
| | | 0.03 | | |
| 4 | 7 | 99.83 | 98.22 | 75.5 |
| | | 0.04 | | |

### Example 7

### Preparation of Eslicarbazepine Acetate

Acetyl chloride (6.5 g, 0.0828 mol) in dichloromethane (50 ml) was added dropwise to a stirred suspension of (10S)-10-hydroxy-10,11-dihydro-5H-dibenzo[b,f]azepine-5-carboxamide (15.05 g, 0.0589 mol) and pyridine (7.05 g, 0.0883 mol) in dichloromethane (250 ml) at 25° C. to 30° C. The reaction mixture was cooled to 15° C. to 20° C. After stirring at 15° C. to 20° C. for 30 minutes, the reaction mixture was stirred for 2 hours at 25° C. to 30° C. After completion of the reaction, the reaction mixture was washed with aqueous sulfuric acid (150 mL), followed by washing with saturated aqueous sodium chloride solution (150 mL) and deionised water (150 mL). The organic layer was divided into two parts by Volume/Volume.

### Part A:

The organic layer obtained above was concentrated to 25 mL at about 35° C. to 40° C. under reduced pressure and was cooled to 15° C. to 20° C. Hexane (25.0 mL) was charged dropwise under stirring. The reaction mixture was stirred for 1 hour at 15° C. to 20° C. The obtained crystalline product was filtered and washed with hexane (7.5 mL). The product was suction dried and was dried under vacuum at 50° C. to 55° C. for 12 hours to obtain titled compound.

### Yield: 5.5 g (63%)

Chemical Purity: 99.84% (Highest individual impurity: 0.04%)

### Chiral Purity: 98.48%

The results of experiments performed by varying the reaction conditions such as the quantity of solvent (dichloromethane and hexane) for the preparation of eslicarbazepine acetate following the procedure of above example is summarized in the table below:

| Volume of | Volume of | Purity/ | | |
|---|---|---|---|---|
| dichloromethane | hexane (times | Highest | Chiral | |
| (times with respect to | with respect to | individual | Purity | Yield |
| eslicarbazepine) | eslicarbazepine) | impurity (%) | (%) | (%) |
| 4 | 8 | 99.87 | 94.51 | 91 |
| | | 0.02 | | |
| 4 | 6 | 99.81 | 97.04 | 81 |
| | | 0.05 | | |
| 3.3 | 3.3 | 99.84 | 98.34 | 63 |
| | | 0.04 | | |

### Part B:

The organic layer obtained above was concentrated to 30 mL at about 35° C. to 40° C. under reduced pressure and was cooled to 15° C. to 2° C. Toluene (75.0 mL) was charged dropwise under stirring. The reaction mixture was stirred for 1 hour at 15° C. to 20° C. The obtained crystalline product was filtered and washed with toluene (7.5 mL). The product was suction dried and was dried under vacuum at 50° C. to 55° C. for 12 hours to obtain titled compound.

### Yield: 5.15 g (58.92%).

Purity: 99.85% (Highest individual impurity: 0.05%)

### Chiral Purity: 98.82%

The results of experiments performed by varying the reaction conditions such as the quantity of solvent (dichloromethane and toluene) for the preparation of eslicarbazepine acetate following the procedure of above example is summarized in the table below:

| Volume of | | | | |
|---|---|---|---|---|
| dichloromethane | Volume of Toluene | | Chiral | |
| (times with respect to | (times with respect to | Purity | purity | Yield |
| eslicarbazepine) | eslicarbazepine) | (%) | (%) | (%) |
| 4 | 10 | 99.85 | 98.82 | 58.92 |
| 4 | 6 | 98.62 | 98.38 | 67.5 |

### Example 8

### Preparation of (10S)-10-Hydroxy-10,11-dihydro-5H-dibenzo[b,f]azepine-5-carboxamide

Pyridine (58.3 g, 0.737 mol) and 4-dimethylaminopyridine (2.9 g, 0.0237 mol) were added to a stirred suspension of racemic-10-hydroxy-10,11-dihydro-5H-dibenzo[b,f]azepine-5-carboxamide (150 g, 0.59 mol) in dichloromethane (1.5 l) at 25° C. to 30° C. Diacetyl-L-tartaric acid anhydride (159.3 g, 0.737 mol) was added after stirring at 25° C. to 30° C. for 15 minutes. The reaction mixture was stirred at 25° C. to 30° C. for 2 hours whereupon deionised water (1.05 l) was added dropwise and the resulting reaction mixture was stirred for 2 hours at 15° C. to 20° C. The precipitated solid was filtered off, washed with deionised water (150 mL) and suction dried for 15 minutes.

Aqueous sodium hydroxide (43 g, 1.075 mol in 350 mL deionised water) was added dropwise to a stirred suspension of (10S)-10-hydroxy-10,11-dihydro-5H-dibenzo[b,f]azepine-5-carboxamide tartarate and water (350.0 mL) obtained above at 15° C. to 20° C. The reaction mixture was stirred at 25° C. to 30° C. for 15 minutes and pH was adjusted to 7.2 using dilute Hydrochloric acid (30 mL concentrated Hydrochloric acid in 100 mL deionised water). The resulting solution was stirred at 25° C. to 30° C. for 30 minutes and was cooled to 5° C. to 10° C. and stirred for 3 hours. The precipitated solid was filtered off, washed with deionised water (100 mL) and dried at 50° C. to 55° C. for 12 hours to obtain the titled compound.

### Yield: 60 g (80.0%)

### Chiral Purity: 87.2%

### Example 9

### Purification of (10S)-10-Hydroxy-10,11-dihydro-5H-dibenzo[b,f]azepine-5-carboxamide

Pyridine (20.9 g, 0.264 mol) and 4-dimethylaminopyridine (1.0 g, 0.0081 mol) were added to a stirred suspension of (10S)-10-hydroxy-10,11-dihydro-5H-dibenzo[b,f]azepine-5-carboxamide (54 g, 0.212 mol) [Chiral purity: 87.21%] and dichloromethane (540 mL) at 25° C. to 30° C. After stirring at 25° C. to 30° C. for 15 minutes, diacetyl-L-tartaric acid anhydride (57.4 g, 0.266 mol) was added to it. The reaction mixture was stirred at 25° C. to 30° C. for 2 hours whereupon deionised water (380 mL) was added dropwise and the resulting reaction mixture was further stirred for 2 hours at 15° C. to 20° C. The precipitated solid was filtered off, washed with deionised water (55 mL) and suction dried for 15 minutes.

Aqueous sodium hydroxide (32 g sodium hydroxide, 0.8 mol in 155 mL deionised water) was added dropwise to a stirred suspension of (10S)-10-hydroxy-10,11-dihydro-5H-dibenzo[b,f]azepine-5-carboxamide tartarate and deionised water (128.0 mL) at 20° C. to 25° C. The reaction mixture was stirred at 25° C. to 30° C. for 15 minutes and was adjusted to pH 6.8 to 7.2 using dilute hydrochloric acid (30 mL concentrated hydrochloric acid in 100 mL deionised water). The resulting solution was stirred at 5° C. to 10° C. for 3 hours. The precipitated solid was filtered off, washed with deionised water (50 mL) and dried at 50° C. to 55° C. for 12 hours to obtain the titled compound.

### Yield: 43.5 g (80.55%)

### Chemical Purity: 98.95%

### Chiral Purity: 96.72%

### Example 10

### Preparation of Eslicarbazepine Acetate

Acetyl chloride (18.4 g, 0.234 mol) in dichloromethane (100 mL) was added dropwise to a stirred suspension of (10S)-10-hydroxy-10,11-dihydro-5H-dibenzo[b,f]azepine-5-carboxamide (35.0 g, 0.13 mol) [Chiral purity: 96.72%] and pyridine (21.3 g, 0.269 mol) in dichloromethane (600 mL) at to 0° C. to 5° C. After stirring at 0° C. to 5° C. for 30 minutes, the reaction mixture was further stirred for 2 hours at 25° C. to 30° C. After completion of the reaction, the reaction mixture was washed with saturated aqueous sodium chloride solution (350 mL), followed by washing with deionised water (150 mL). The organic layer was concentrated at about 40° C. to 45° C. under reducing pressure. Hexane (70 mL) was added to the above solid and stirred for 30 minutes at 25° C. to 30° C. The precipitated solid was filtered off, washed with hexane (10 mL) and dried at 50° C. to 55° C. for 6 hours to obtain the titled compound.

### Yield: 35.1 g (86.07%)

### Chemical Purity: 96.01%

### Chiral Purity: 98.32%

### Example 11

### Purification of Eslicabazepine Acetate

Ethyl acetate (21.0 mL) was added dropwise to eslicarbazepine acetate (3.0 g) and dichloromethane (12.0 mL) at 25° C. to 30° C. followed by hexane (9.0 mL) under stirring. The resulting suspension was cooled to 0° C. to 5° C. and stirred for 1 hour at 0° C. to 5° C. The crystalline product was filtered and washed with hexane (3.0 mL). The product was suction dried and dried under vacuum at 45° C. to 50° C. for 13 hours to obtain the titled compound.

### Yield: 2.46 g (82%)

Chiral purity: 99.82%

### Example 12

### Preparation of Eslicarbazepine Acetate

Acetic anhydride (1.6 g, 0.0153 mol) was added to a stirred suspension of (10S)-10-hydroxy-10,11-dihydro-5H-dibenzo[b,f]azepine-5-carboxamide (3.0 g, 0.0117 mol) [Chiral Purity: 90% to 94%], acetone (22.5 mL), and 4-dimethyl amino pyridine (0.086 g, 0.0007 mol) at 25° C. to 30° C. The reaction mixture was stirred at 25° C. to 30° C. for 2 hours. After completion of the reaction, deionised water (45 mL) was added dropwise and the mixture was stirred for 3 hours at 25° C. to 30° C.

The product was filtered and washed with deionised water (15 mL). The product was suck dried and further dried under vacuum at 50° C. to 55° C. for 12 hours to obtain the titled compound.

### Yield: 2.4 g (68.9%)

### Chemical Purity: 99.83%

### Chiral Purity: 99.66%

The results of experiments performed by varying the reaction conditions such as the quantity of solvent (acetone and water) for the preparation of eslicarbazepine acetate following the procedure of the above example is summarized in the table below:

| Volume of | Volume | | | | |
|---|---|---|---|---|---|
| acetone | of water | | | | |
| (times with | (times with | Tem- | | Chiral | |
| respect to | respect to | perature | Yield | Purity | Purity |
| eslicarbazepine) | eslicarbazepine) | (° C.) | (%) | (%) | (%) |
| 6.25 | 12.5 | 15 | 69.96 | 99.79 | 99.77 |
| 6.25 | 17.5 | 15 | 79.31 | 99.64 | 99.69 |
| 8.75 | 17.5 | 15 | 69.82 | 99.62 | 99.92 |
| 7.5 | 15 | 20 | 69.96 | 99.66 | 99.83 |
| 6 | 18 | 0-5 | 84.48 | 98.38 | 99.85 |
| 6 | 18 | 15 | 79.31 | 99.71 | 99.85 |
| 6 | 18 | 22-25 | 77.59 | 99.8 | 99.87 |
| 5 | 15 | 20 | 76.72 | 99.46 | 99.79 |
| 6 | 18 | 15 | 70.69 | 99.58 | 99.59 |
| 5 | 18 | 12 | 86.2 | 97.8 | 99.92 |

### Example 13

### Preparation of Eslicarbazepine Acetate

Acetic anhydride (1.6 g, 0.0153 mol) was added to a stirred suspension of (10S)-10-hydroxy-10,11-dihydro-5H-dibenzo[b,f]azepine-5-carboxamide (3.0 g, 0.0117 mol; Chiral purity: 90%-94%), dichloromethane (19 mL), and 4-dimethylaminopyridine (0.086 g, 0.0007 mol) at 25° C. to 30° C. The reaction mixture was stirred at 25° C. to 30° C. for 2 hours. After completion of the reaction, cyclohexane (48 mL) was charged dropwise and the mixture was stirred for 3 hours at 25° C. to 30° C.

The product was filtered and washed with cyclohexane (15 mL). The product was suck dried and further dried under vacuum at 50° C. to 55° C. for 12 hours to obtain the titled compound.

### Yield: 2.6 g (75%)

Chemical Purity: 99.95% (Highest individual impurity: 0.02%)

### Chiral Purity: 99.14%

### Example 14

### Preparation of Oxcarbazepine

To a stirred suspension of 10-hydroxy-10,11-dihydro-5H-dibenzo[b,f]azepine-5-carboxamide (2.0 g, 0.0078 mol) in dichloromethane (10 mL), TEMPO (13 mg, 0.000078) was charged at 25° C. to 30° C. After stirring at 25° C. to 30° C. for 15 minutes, the reaction mixture was cooled to 0° C. to 5° C. and sodium hypochlorite solution (12.0 mL) (adjusted pH=9.7 using 10% aqueous NaHCO₃) was charged dropwise. The reaction mixture was stirred at 0° C. to 5° C. for 1 hour. The organic layer was separated and washed with deionised water (20 mL) and concentrated at about 40° C. to 45° C. under reducing pressure. The solid was stirred with toluene (5 mL) and filtered. The solid obtained was stirred with dimethylformamide (12 mL) at 60° C. to 65° C. After stirring at 60° C. to 65° C. for 15 minutes, the reaction mixture was cooled to 20° C. to 25° C. Deionised water (72 mL) was charged and the mixture was further stirred for 30 minutes. The solid was filtered, washed with deionised water (10 mL) and dried at 60° C. to 65° C. under vacuum for 8 hours to obtain the titled compound.

### Yield: 1.3 g (65.5%)

Chemical Purity: 90.4%

1. A process for the preparation of oxcarbazepine of Formula 1; 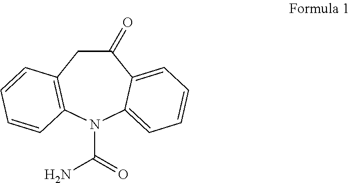 [SMILES: NC(=O)N1c2ccccc2CC(=O)c2ccccc21]
the process comprising the oxidation of 10-hydroxy-10,11-dihydro-5H-dibenzo[b,f]azepine-5-carboxamide (Formula 2) 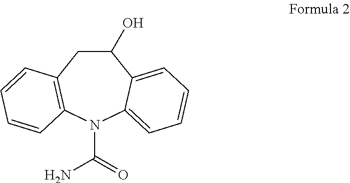 [SMILES: NC(=O)N1c2ccccc2CC(O)c2ccccc21]
with an oxidizing agent, wherein the oxidizing agent is a mixture of (2,2,6,6-Tetramethyl-piperidin-1-yl)oxyl (TEMPO) and sodium hypochlorite.

2. A process according to claim 1, wherein the oxidation of the compound of Formula 2 is performed in one or more solvents.

3. A process according to claim 2, wherein the solvent is water, esters, halogenated hydrocarbons, ketones, ethers, polar aprotic solvents, or mixtures thereof.

4. A process according to claim 3, wherein the ester is ethyl acetate, n-propyl acetate, isopropyl acetate, or n-butyl acetate.

5. A process according to claim 3, wherein the halogenated hydrocarbon is dichloromethane, chloroform, or 1,2-dichloroethane.

6. A process according to claim 3, wherein the ketone is acetone or methyl ethyl ketone.

7. A process according to claim 3, wherein the ether is diethyl ether or tetrahydrofuran.

8. A process according to claim 3, wherein the polar aprotic solvent is N,N-dimethylformamide, N,N-dimethylacetamide, dimethylsulphoxide, acetonitrile or N-methylpyrrolidone.

9. A process according to claim 1, wherein the oxidation of the compound of Formula 2 is performed at a temperature of 0° C. to 50° C.

10. A process for the preparation of eslicarbazepine acetate of Formula A 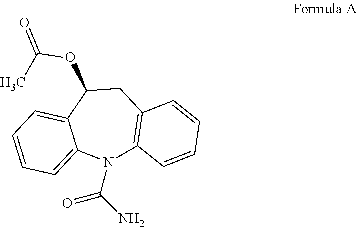 [SMILES: CC(=O)O[C@H]1Cc2ccccc2N(C(N)=O)c2ccccc21]
which comprises the steps of:
a) providing a solution of eslicarbazepine acetate in dichloromethane;
b) combining the solution obtained in step a) with a solvent selected from the group consisting of cyclohexane, hexane, toluene or a mixture thereof or with a mixture of ethyl acetate and hexane; and
c) isolating eslicarbazepine acetate.

11. A process according to claim 10, wherein step a) includes dissolving eslicarbazepine acetate in dichloromethane or using a solution of eslicarbazepine acetate in dichloromethane from a previous processing step of eslicarbazepine acetate in dichloromethane.

12. A process according to claim 10, wherein the volume of dichloromethane is about 2 times to about 15 times the weight of eslicarbazepine.

13. A process according to claim 10, wherein step b) involves combining the solution obtained in step a) with a solvent selected from the group consisting of cyclohexane, hexane, toluene, or a mixture thereof.

14. A process according to claim 10, wherein step b) involves combining the solution obtained in step a) with a mixture of ethyl acetate and hexane.

15. A process according to claim 10, wherein step b) involves adding the solvent at about 10° C. to 20° C.

16. A process for the preparation of eslicarbazepine acetate of Formula A 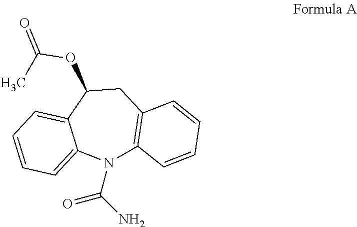 [SMILES: CC(=O)O[C@H]1Cc2ccccc2N(C(N)=O)c2ccccc21]
comprising the steps of:
a) providing a mixture of eslicarbazepine acetate in acetone;
b) combining the mixture obtained in step a) with water; and
c) isolating eslicarbazepine acetate.

17. A process according to claim 16, wherein step a) includes dissolving or suspending eslicarbazepine acetate in acetone at a temperature of about 25° C. to 40° C. optionally under stirring, or using a solution from a previous processing step of eslicarbazepine acetate in acetone.

18. A process according to claim 16, wherein step b) involves combining the mixture obtained in step a) with water at about 10° C. to 40° C.

19. A process according to claim 16, wherein the volume of water is about 5 times to about 20 times the weight of eslicarbazepine.

20. A process for the preparation of eslicarbazepine acetate of Formula A 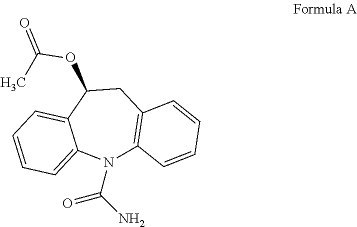 [SMILES: CC(=O)O[C@H]1Cc2ccccc2N(C(N)=O)c2ccccc21]
comprising the steps of:
a) treating (10S)-10-hydroxy-10,11-dihydro-5H-dibenzo[b,f]azepine-5-carboxamide with an acylating agent in acetone;
b) combining the mixture obtained in step a) with water; and
c) isolating eslicarbazepine acetate.

21. A process according to claim 20, wherein step a) of treating (10S)-10-hydroxy-10,11-dihydro-5H-dibenzo[b,f]azepine-5-carboxamide with an acylating agent in acetone is performed in the presence of a catalyst at a temperature of about 20° C. to 40° C.

22. A process according to claim 20, wherein the acylating agent is acetyl chloride or acetic anhydride.

23. A process according to claim 20, wherein the molar ratio of the acylating agent is in the range of 0.5 to 1.5.

24. A process according to claim 21, wherein the catalyst is an organic base.

25. A process according to claim 21, wherein the molar ratio of the catalyst is preferably in the range of 0.05 to 0.2.

26. A process according to claim 20, wherein step b) involves combining the solution obtained in step a) with water at about 10° C. to 40° C.

27. A process according to claim 20, wherein the volume of water is about 5 times to about 20 times more than the weight of eslicarbazepine.
